(19) United States
(12) Patent Application Publication
(10) Pub. No.: US 2025/0306152 A1
(43) Pub. Date: Oct. 2, 2025

(54) Elastography Apparatus for MR Elastography

(71) Applicant: Siemens Healthineers AG, Forchheim (DE)

(72) Inventors: Julian Richter, Hessdorf (DE); Manuel Stich, Schwandorf (DE)

(73) Assignee: Siemens Healthineers AG, Forchheim (DE)

(21) Appl. No.: 19/090,636

(22) Filed: Mar. 26, 2025

(30) Foreign Application Priority Data
Mar. 26, 2024 (DE) 10 2024 202 865.7

(51) Int. Cl. G01R 33/563; A61B 5/00; A61B 5/055

(52) U.S. Cl. CPC G01R 33/56358; A61B 5/0051; A61B 5/055; A61B 5/6831

(57) ABSTRACT

The disclosure relates to an elastography apparatus designed to support magnetic resonance elastography, including an oscillation generator, a fixation apparatus designed to fix the oscillation generator to an object under examination, a pressure sensor designed to detect a contact pressure acting between the oscillation generator and the object under examination, and an output unit designed to output the detected contact pressure.

### TECHNICAL FIELD

The disclosure relates to an elastography apparatus for a magnetic resonance device and a method for preparing an MR elastography using the elastography apparatus.

### BACKGROUND

In a magnetic resonance device, the body of an object under examination, in particular a patient, is typically exposed to a relatively high main magnetic field, for example, 1.5 or 3 tesla, using a main magnet. During magnetic resonance (MR) imaging, gradient pulses are applied using a gradient coil unit. In addition, high-frequency radiofrequency (RF) pulses, in particular excitation pulses, are emitted via a radiofrequency antenna unit using suitable antenna equipment, which causes the nuclear spins of certain atoms, resonantly excited by these RF pulses, to tilt by a defined flip angle relative to the field lines of the main magnetic field. When the nuclear spins relax, radiofrequency signals, known as magnetic resonance signals, are emitted, which are received by suitable radiofrequency antennas and then undergo further processing. The desired image data can then be reconstructed from the raw data acquired in this way. For a particular measurement, a specific magnetic resonance (MR) control sequence must be transmitted, consisting of a series of radiofrequency pulses, such as excitation and refocusing pulses, as well as gradient pulses appropriately coordinated thereto, in different gradient axes along different spatial directions. Readout windows are set in a time-coordinated manner, defining the periods during which the induced magnetic resonance signals are captured.

MR elastography (MRE) utilizes the fact that the phase of the magnetic resonance signals changes as a result of mechanical waves acting on the object under examination. The extent of this change depends on the displacement (i.e., shift from the resting position) of the tissue due to the mechanical waves. Thus, information about certain mechanical parameters of the tissue, such as elasticity, can be derived from MR phase images, i.e., images that depict the phase of the nuclear magnetization. MRE is, therefore, a non-invasive method for quantifying the elasticity and stiffness of tissue. In addition to a conventional magnetic resonance device, MRE requires an oscillation generator to deliver the mechanical waves, especially in the examination area of the object under examination. Precise positioning of the oscillation generator on the body surface is essential to ensure that the mechanical waves penetrate the organ under examination in a defined direction. Less than ideal positioning of the oscillation generator can reduce the quality of the MRE data and, in particular, affect the statistical confidence of shear modulus quantification.

### SUMMARY

An object of the disclosure is to provide an elastography apparatus for MRE that is particularly easy to position and robust, as well as comfortable for the patient. This object is achieved by the features of the independent claims. Advantageous aspects are described in the dependent claims.

The disclosed elastography apparatus, which is designed to support magnetic resonance elastography of an object under examination, comprises:
an oscillation generator,
a fixation apparatus designed to secure the oscillation generator to an object under examination,
a pressure sensor designed to detect a contact pressure acting between the oscillation generator and the object under examination and/or between the fixation apparatus and the object under examination, and
an output unit designed to output the detected contact pressure.

The elastography apparatus, according to the disclosure, is preferably designed to perform an MRE in combination with a magnetic resonance device. The oscillation generator is designed to emit mechanical oscillations, particularly with shear and compression components. The oscillation generator is preferably designed as a passive oscillation generator. The oscillation generator can be designed as a gravitational oscillation generator, in particular, according to US20230305090A1. During the MRE, the oscillation generator is to be arranged on the body surface of the object under examination at the examination area. The fixation apparatus is designed to secure the oscillation generator to a region on the body surface corresponding to the desired examination area. The fixation apparatus can be designed as a belt and/or band and/or strap, for example.

The fixation apparatus is preferably suitable for a variety of objects under examination, in particular having different body sizes and/or chest circumferences. The fixation apparatus can be specifically designed for an examination area, for example the liver. The fixation apparatus is preferably reusable, reversibly detachable, and/or reclosable. The fixation apparatus is preferably designed such that contact pressure acting between the oscillation generator and the object under examination can be variably adjusted and/or changed. For this purpose, the fixation apparatus can, for example, comprise a length-adjustable belt and/or an incrementally adjustable closure unit.

The pressure sensor is typically designed to quantify and/or qualitatively determine the contact pressure, particularly acting on the body surface of the object under examination and/or between the fixation apparatus and the object under examination. The output unit is designed to provide a user with a signal relating to the contact pressure detected. In particular, the output unit can provide the user who is arranging the elastography apparatus on an object under examination during preparation for an MRE with feedback regarding the contact pressure and, thus, the tightness of the oscillation generator on the body surface and/or comfort for the object under examination.

The outputting of the detected contact pressure provides an objective guideline and/or instruction for positioning the elastography apparatus on the object under examination. This allows the user to change the contact pressure by varying the fixation apparatus and, in particular, aligning it to a defined reference value. In addition, the elastography apparatus, according to the disclosure, ensures an adequate contact pressure that can be consistently applied across multiple objects under examination and/or to the same object under examination, thus facilitating MRE with consistent and reproducible quality. This also reduces the risk of having to repeat an MRE examination due to insufficient quality and/or inaccurate transmission of the mechanical waves.

An aspect of the elastography apparatus provides that:
the pressure sensor comprises a manometer, a compressible pad, and a connecting unit linking the compressible pad to the manometer, and
the compressible pad is positioned between the fixation apparatus and the object under examination when the oscillation generator is arranged on the object under examination.

The connecting unit is preferably of airtight and/or watertight design. The compressible pad typically comprises a covering that encloses a medium. The compressible pad, in particular the covering, is preferably flexibly moldable. The compressible pad is typically flat and/or has a height of less than 3 cm, preferably less than 2 cm, particularly preferably less than 1 cm. The medium may comprise a gas, particularly air, or a liquid, such as water.

The manometer is typically designed to quantify a pressure originating from the compressible pad. The manometer is typically located at least 1 m away from the compressible pad. The manometer can be part of an external component of the elastography apparatus. For example, a vibration-generating unit required for controlling the oscillation generator can also be designed as part of the external component.

When the elastography apparatus is arranged on an object under examination, the compressible pad typically comes into contact with the body surface and/or the clothing of the object under examination. The compressible pad can be arranged at least partially between the fixation apparatus and the object under examination and/or between the fixation apparatus and the oscillation generator and/or between the oscillation generator and the object under examination.

The compression of the compressible pad typically corresponds to the degree of contact pressure, in particular, acting between the oscillation generator and the object under examination. The use of a connecting unit allows spatial distancing between the manometer and the compressible pad, which is particularly advantageous for MRE due to the usual positioning of the oscillation generator, and thus of the compressible pad, in a strong main magnetic field, as the manometer can be positioned at a distance from the main magnetic field. Moreover, the manometer can be positioned within sight of a user, which allows for convenient operation of the elastography apparatus and monitoring of the contact pressure. This aspect of the pressure sensor allows for robust detection of the contact pressure, thus providing precise feedback to the user as to whether the fixation of the oscillation generator is appropriate, comfortable, and/or robust.

One aspect of the elastography apparatus provides that
the oscillation generator has a first side designed to be arranged on the object under examination, and
the compressible pad is arranged on the first side of the oscillation generator, and when the oscillation generator is arranged on the object under examination, it is positioned between the first side of the oscillation generator and the object under examination.

The compressible pad can be fixed, in particular detachably fixed, to the first side of the oscillation generator. The compressible pad preferably comprises and/or encloses a medium that is particularly effective at transmitting mechanical waves produced by the oscillation generator. The compressible pad typically has a flexible surface. Especially when positioned between the oscillation generator and the body surface of the object under examination, the compressible pad can indirectly create a particularly large contact area between the oscillation generator and the object under examination. The compressible pad can also conform to the shape of the body surface of the object under examination. This allows integrated measurement of the contact pressure, while simultaneously improving comfort for the object under examination, and better transmission of the mechanical waves from the oscillation generator to the examination area.

An aspect of the elastography apparatus provides that the connecting unit comprises a tube and/or a hose. The connecting unit preferably includes plastic and/or synthetic material. A hose is typically flexible, allowing for a non-rigid connection between the compressible pad and the manometer. This allows easy measurement of the contact pressure.

An aspect of the elastography apparatus provides that the fixation apparatus comprises an elastic belt. In particular, an elastic belt allows the oscillation generator to be arranged on the object under examination in a manner that is comfortable for the object under examination and easily fixed in position. In addition, bands of elastic material are suitable for fixing an oscillation generator to a variety of body circumferences, making such a fixation apparatus universally usable.

An aspect of the elastography apparatus provides that the fixation apparatus comprises a belt and a closure unit, the belt being able to be wrapped around a chest of an object under examination, and the closure unit being designed to secure the belt wrapped around the chest of an object under examination.

The belt can be designed as an elastic belt and/or as a band. The closure unit can be designed as a buckle and/or a snap buckle and/or a clasp. Consequently, the fixation apparatus, according to this aspect, can correspond to a buckle-fastened strap. The closure unit is preferably designed such that the length of the belt is variably adjustable and/or the length of a portion of the belt to be wrapped around the chest is selectable. A fixation apparatus of this kind is particularly cost-effective and flexibly usable.

An aspect of the elastography apparatus provides that the oscillation generator is arranged on the side of the belt facing the chest. This ensures particularly good transmission of the mechanical waves.

An aspect of the elastography apparatus provides that the pressure sensor is designed to quantify a tensile force acting on the closure unit and/or the belt and/or the fixation apparatus. The fixation apparatus can comprise an elastic belt. The tensile force typically correlates linearly, preferably approximately linearly, with the contact pressure. The pressure sensor can, for example, be designed analogously to a luggage weigher and/or comprise a coil spring. A pressure sensor of this kind can be easily incorporated in the fixation apparatus and/or into a band and/or into a belt and robustly quantify the tensile force acting on the fixation apparatus.

An aspect of the elastography apparatus provides that the output unit includes a scale for measuring the tensile force. In particular, the scale can be designed as an elastic scale. The scale, in particular the output unit, is preferably integrated in the closure unit. The scale makes it possible to quantify the contact pressure and, in particular, any deviation of the currently applied contact pressure from a reference value. Such a scale is typically robust and intuitively integratable.

An aspect of the elastography apparatus provides that the output unit is designed to output a reference value for the contact pressure and/or the difference between the detected contact pressure and a reference value for the contact pressure.

The reference value typically corresponds to a target value for the contact pressure and/or an ideal contact pressure. If the contact pressure between the oscillation generator and the body surface corresponds to the reference value, a secure hold of the oscillation generator, homogeneous and efficient penetration of the mechanical waves into the examination area and comfort for the object under examination are typically ensured. In particular, the reference value typically corresponds to the value at which displacement due to movement of the object under examination and/or vibrations inherent in operation of the oscillation generator are eliminated. The reference value may also include a range of values, in particular, a reference range. If, in addition to displaying the detected contact pressure, the output unit indicates a reference value and/or a difference between the detected contact pressure and this reference value, the user can position the fixation unit with appropriate contact pressure before initiating the MRE measurement, in order to achieve the reference value for the contact pressure. Arranging the oscillation generator with a contact pressure corresponding to the reference value reduces the likelihood of having to repeat an MRE measurement, especially due to the low quality of the transmitted mechanical waves.

An aspect of the elastography apparatus provides that the difference between the detected contact pressure and a reference value for the contact pressure is output in a classified manner. As a means of classifying the detected contact pressure, the output unit is preferably designed to indicate contact pressure ranges in which the mechanical waves are transmitted only inadequately or not at all. For example, an acceptable value range for the contact pressure, corresponding to the reference value, can be indicated differently from the contact pressure range, indicating a 20% reduction in quality. Another contact pressure range likely to result in more than a 20% reduction in quality can be indicated differently again from the other two categories. The distinction between the categories can be indicated by color and/or using numbers. This allows a particularly simple iterative approximation of the actual contact pressure to the reference value by user adjustment of the fixation apparatus.

An aspect of the elastography apparatus provides that the output unit comprises a visual display unit. A visual display can include a scale, for example. A manometer is typically visually readable. A scale incorporated in the fixation apparatus and/or closure unit is also visually readable. This aspect of the output unit is typically arranged within sight of the fixation apparatus so that a user can read the detected contact pressure when adjusting the fixation apparatus.

An aspect of the elastography apparatus provides that the output unit comprises an audio signal unit. According to this aspect, the output unit preferably includes a speaker. When a reference value is reached, a tone can be emitted, for example. Likewise, a tone of a defined pitch and/or of a defined length can be emitted depending on the difference and/or deviation between the detected contact pressure and the reference value. This provides a particularly easy means of changing the contact pressure by adjusting the fixation apparatus without having to read a scale.

An aspect of the elastography apparatus provides that the elastography apparatus additionally comprises a vibration-generating unit and a connector, the latter connecting the vibration-generating unit to the oscillation generator. The vibration-generating unit can include a stepper motor. The stepper motor can be designed, for example, as described in US20230296708A1. The connector can comprise a rotary feed. This aspect allows for flexible control of the oscillation generator.

The disclosure is also based on a method for preparing and/or performing an MR elastography of an object under examination using an elastography apparatus according to the disclosure, involving positioning of the elastography apparatus according to the following steps:

1. Fixing the oscillation generator to a surface of the body of the object under examination with an initial contact pressure using the fixation apparatus, preferably by attachment of the oscillation generator and the fixation apparatus by medical personnel, in particular a user. In this process, the personnel will apply the oscillation generator and fix it in position by means of the fixation apparatus based on their experience, wherein an initial contact pressure is generated between the oscillation generator and the body surface of the object under examination.

2. Detecting an initial contact pressure acting between the oscillation generator and the object under examination using the pressure sensor. The recorded initial contact pressure is preferably output by means of the output unit.

3. Determining a deviation of the detected initial contact pressure from a contact pressure reference value indicated by the output unit. The output unit typically also indicates a reference value for the contact pressure and/or a deviation of the detected initial contact pressure from a reference value for the contact pressure. Alternatively, the personnel may be aware of a reference value for the contact pressure and the personnel can compare the detected initial contact pressure with the reference value. The deviation can be determined and/or output quantitatively and/or qualitatively.

4. Modifying the fixation apparatus with respect to the contact pressure applied to the oscillation generator depending on the deviation of the contact pressure. In particular, if the deviation is greater than a threshold value for the deviation of the contact pressure, a user can adjust the fixation apparatus and/or the closure unit such that the deviation between the detected contact pressure and the reference value is reduced. This can be performed iteratively until the reference value is attained and/or the deviation from the reference value is less than a threshold value for the deviation of the contact pressure.

An elastography apparatus arranged according to the disclosure is adequately fixed and guarantees a secure hold on the object under examination for the duration of the MRE examination.

Embodiments of the method, according to the disclosure, are designed analogously to the aspects of the elastography apparatus according to the disclosure. The advantages of the method according to the disclosure essentially correspond to the advantages of the elastography apparatus according to the disclosure, which are detailed above. Features, advantages, or alternative aspects mentioned herein can also be applied to the method and vice versa.

### DESCRIPTION OF THE DRAWINGS

Further advantages, features, and details of the disclosure will emerge from the exemplary aspects described below and the accompanying drawings in which:

FIG. 1 schematically illustrates a first aspect of an elastography apparatus according to the disclosure,

FIG. 2 schematically illustrates a second aspect of an elastography apparatus according to the disclosure,

FIG. 3 schematically illustrates a fixation apparatus with a pressure sensor, and

FIG. 4 shows a flowchart of an aspect of a method according to the disclosure.

### DETAILED DESCRIPTION

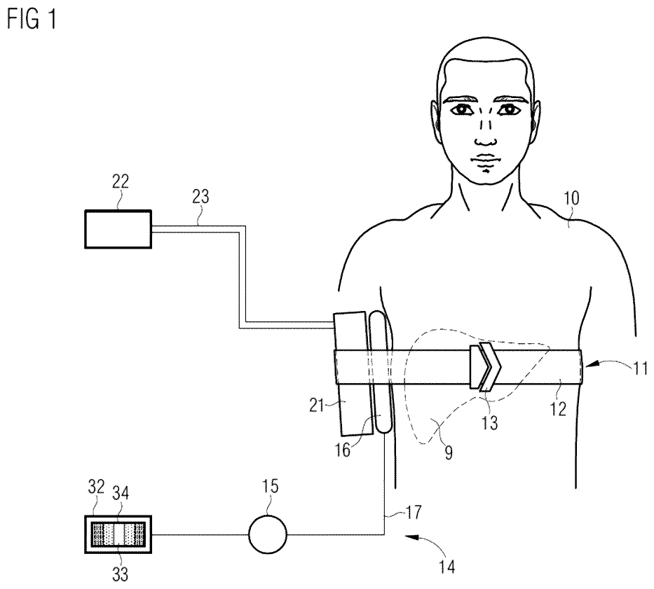

FIG. 1 schematically illustrates a first aspect of an elastography apparatus according to the disclosure. The elastography apparatus comprises an oscillation generator 21 and a fixation apparatus 11. The fixation apparatus 11 is designed to secure the oscillation generator to an object under examination 10. In the aspect shown, the object under examination 10 has a liver 9 that is the target of the MRE examination. The fixation apparatus 11 is arranged such that the oscillation generator 21 can transmit mechanical waves in the direction, particularly along the longitudinal direction, of the liver 9.

The elastography apparatus additionally comprises a pressure sensor 14 which is designed to detect a contact pressure acting between the oscillation generator 21 and the object under examination 10 and thus also acting between the fixation apparatus 11 and the object under examination 10. The elastography apparatus also comprises an output unit 32, designed in this aspect as a visual display unit 34, which is used to output, in particular to indicate, the detected contact pressure.

The output unit specifically indicates a reference value 33 for the contact pressure. The additional display of the detected contact pressure indicates the difference from the reference value 33 for the contact pressure, providing a qualitative and/or quantitative measure of the deviation from the reference value 33. For this purpose, the visual display unit 34 comprises a color scale and/or grayscale, wherein the center of the color scale corresponds to the reference value 33 for the contact pressure.

In the aspect shown, the pressure sensor 14 comprises a compressible pad 16, which is positioned between the oscillation generator 21 and the body surface of the object under examination 10. For this purpose, the compressible pad 16 is arranged on the side of the oscillation generator 21 facing the object under examination 10 and is preferably fixed in a reversibly detachable manner. In an alternative arrangement (not shown in detail here) of the compressible pad 16, the compressible pad 16 is arranged between the fixation apparatus 11 and the object under examination 10, in direct contact with the fixation apparatus 11, and in direct contact with the object under examination 10. For example, it could be fixed on the side of the object under examination 10 opposite the oscillation generator 21 so that the oscillation generator 21 would be arranged on the right arm side and the compressible pad 16 on the left arm side.

The pressure sensor 14 additionally comprises a manometer 15 and a connecting unit 17, which connects the compressible pad 16 to the manometer 15. The connecting unit 17 can be designed as a tube and/or as a hose, providing, in particular, an airtight connection between the manometer 15 and the compressible pad 16.

The fixation apparatus 11 comprises a belt 12 and a closure unit 13. In the case shown, the belt is wrapped around the chest of the object under examination 10 at the level of the liver 9. The closure unit 13 fastens the belt 12 by interconnecting the two ends of the belt 12. The oscillation generator 21 is arranged between the belt 12 and the chest, i.e., the body surface of the object under examination 10. The belt 12 can be elastic. Alternatively or additionally, the closure unit 13 can be designed such that it is possible to variably adjust the length of the portion of the belt 12 which is wrapped between the closure unit 13, in particular is wrapped around the object under examination 10.

According to this aspect, the elastography apparatus comprises a vibration-generating unit 22, such as a stepper motor and a connector 23. The connector 23 connects the vibration-generating unit 22 to the oscillation generator 21. The oscillation generator 21 is designed as a passive element so that, according to this aspect, the oscillation generator 21 is activated by the vibration-generating unit 22 via the connector 23.

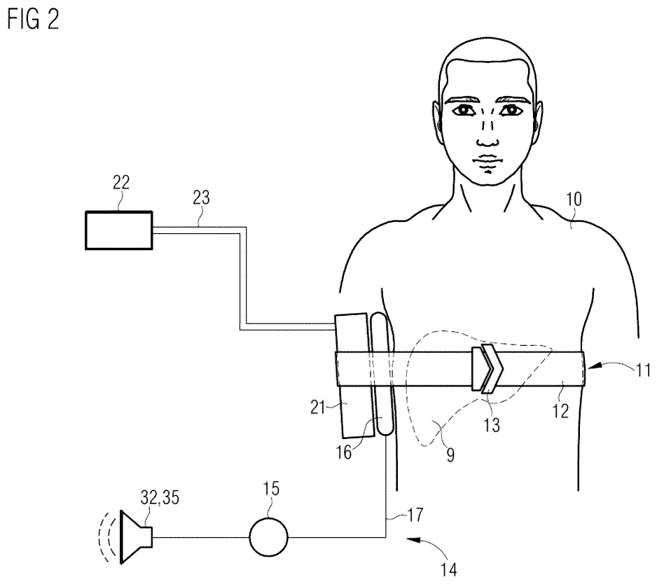

FIG. 2 schematically illustrates a second aspect of an elastography apparatus according to the disclosure. The second aspect differs from the first aspect shown in FIG. 1 in that, instead of the visual display unit 34, the output unit 32 comprises an audio signal unit 35. It is also conceivable for the output unit 32 to comprise both an audio signal unit 35 and a visual display unit 34.

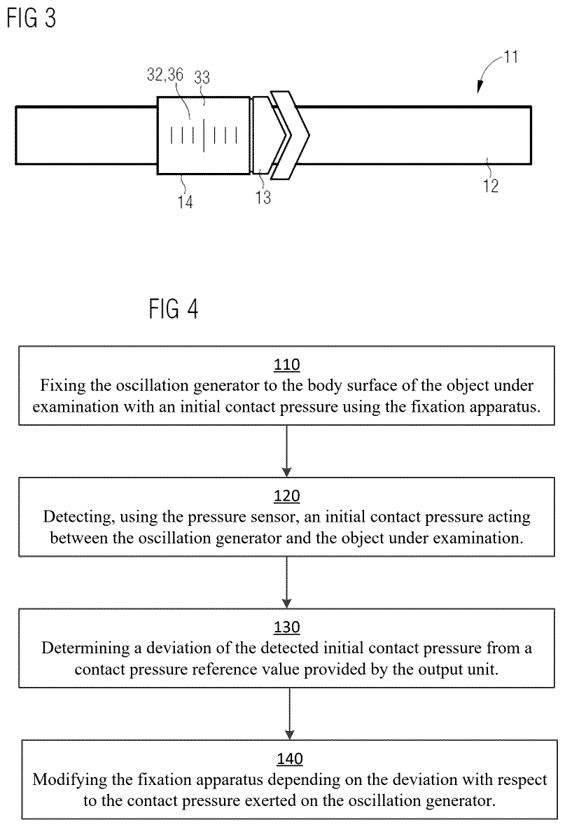

FIG. 3 schematically illustrates an aspect of fixation apparatus 11 with a pressure sensor. As described in FIG. 1, the fixation apparatus 11 comprises a belt 12 and a closure unit 13. The pressure sensor 14 is designed to quantify a tensile force acting on the closure unit 13 and on the belt 12. For this purpose, the pressure sensor 14 can comprise for example, a coil spring. The tensile force scales with the contact pressure; in particular, the tensile force can scale linearly with the contact pressure. The reference value 33 for the contact pressure can, therefore, be specified as a reference value 33 for the tensile force.

The scale 36 incorporated in the output unit 32 relates to the tensile force. The reference value 33 is marked on the scale 36. In particular, the difference between the detected contact pressure and the reference value 33 for the contact pressure is visible on the scale. In addition, the scale 36 allows the detected contact pressure to be classified: for example, a certain area of the scale 36 can be marked, a tensile force within this range corresponding to an accepted contact pressure.

FIG. 4 shows a flowchart of an aspect of a method according to the disclosure for preparing an MR elastography of an object under examination using an elastography apparatus according to the disclosure. The method comprises step 110, which involves fixing the oscillation generator 21 to the body surface of the object under examination 10 with an initial contact pressure using the fixation apparatus 11. The subsequent method step 120 involves detecting, by means of the pressure sensor 14, an initial contact pressure acting between the oscillation generator 21 and the object under examination 10. Step 130 involves determining a deviation of the detected initial contact pressure from a contact pressure reference value provided by the output unit 32. Step 140 involves modifying the fixation apparatus 11 depending on the deviation with respect to the contact pressure exerted on the oscillation generator 21, particularly if the deviation exceeds a threshold value.

Although the aspects of the disclosure have been illustrated and described in detail by the preferred exemplary aspects, the aspects of the disclosure are not limited to the disclosed examples, and other variations will be apparent to persons skilled in the art without departing from the scope of the disclosure. Independent of the grammatical term usage, individuals with male, female, or other gender identities are included within the term.

1. An elastography apparatus designed to support magnetic resonance elastography, comprising:
an oscillation generator;
a fixation apparatus designed to fix the oscillation generator to an object under examination;
a pressure sensor designed to detect a contact pressure acting between the oscillation generator and the object under examination and/or acting between the fixation apparatus and the object under examination; and
an output unit designed to output the detected contact pressure.

2. The elastography apparatus as claimed in claim 1, wherein:
the pressure sensor comprises a manometer, a compressible pad, and a connecting unit connecting the compressible pad to the manometer, and
the compressible pad is positioned between the fixation apparatus and the object under examination when the oscillation generator is arranged on the object under examination.

3. The elastography apparatus as claimed in claim 2, wherein:
the oscillation generator has a first side designed to be arranged on the object under examination, and
the compressible pad is arranged on the first side of the oscillation generator, and when the oscillation generator is arranged on the object under examination, it is positioned between the first side of the oscillation generator and the object under examination.

4. The elastography apparatus as claimed in claim 2, wherein the connecting unit comprises a tube and/or a hose.

5. The elastography apparatus as claimed in claim 1, wherein the fixation apparatus comprises an elastic belt.

6. The elastography apparatus as claimed in claim 1, wherein the fixation apparatus comprises a belt and a closure unit, the belt is placeable around a chest of an object under examination, and the closure unit is designed to fasten the belt placed around the chest of an object under examination.

7. The elastography apparatus as claimed in claim 6, wherein the oscillation generator is arranged on a side of the belt facing the chest.

8. The elastography apparatus as claimed in claim 6, wherein the pressure sensor is designed to quantify a tensile force acting on the closure unit and/or the belt and/or the fixation apparatus.

9. The elastography apparatus as claimed in claim 8, wherein the output unit comprises a scale for measuring the tensile force.

10. The elastography apparatus as claimed in claim 1, wherein the output unit is designed to output a reference value for the contact pressure and/or a difference between the detected contact pressure and a reference value for the contact pressure.

11. The elastography apparatus as claimed in claim 10, wherein the difference between the detected contact pressure and a reference value for the contact pressure is output in a classified manner.

12. The elastography apparatus as claimed in claim 1, wherein the output unit comprises a visual display unit.

13. The elastography apparatus as claimed in claim 1, wherein the output unit comprises an audio signal unit.

14. The elastography apparatus as claimed in claim 1, further comprising:
a vibration-generating unit and a connector connecting the vibration-generating unit to the oscillation generator.

15. A method for preparing an MR elastography of an object under examination using an elastography apparatus as claimed in claim 1, the method comprising:
fixing the oscillation generator to a body surface of the object under examination with an initial contact pressure using the fixation apparatus;
detecting, using the pressure sensor, an initial contact pressure acting between the oscillation generator and the object under examination;
determining a deviation of the detected initial contact pressure from a contact pressure reference value output by the output unit; and
modifying the fixation apparatus with respect to the contact pressure produced on the oscillation generator depending on the deviation.
